(19) United States
(12) Patent Application Publication
(10) Pub. No.: US 2022/0348608 A1
(43) Pub. Date: Nov. 3, 2022

(54) PURIFICATION OF PROTEINS AND VIRAL INACTIVATION

(71) Applicant: MERCK PATENT GMBH, DARMSTADT (DE)

(72) Inventors: Christoph KORPUS, Darmstadt (DE); Supriyadi HAFIZ, Darmstadt (DE); Alexandra KROG, Darmstadt (DE); Romas SKUDAS, Darmstadt (DE)

(73) Assignee: MERCK PATENT GMBH, DARMSTADT (DE)

(21) Appl. No.: 17/765,960

(22) PCT Filed: Oct. 1, 2020
(86) PCT No.: PCT/EP2020/077469

§ 371 Date: Apr. 1, 2022

(30) Foreign Application Priority Data
Oct. 4, 2019 (EP) 19201515.4

(51) Int. Cl. C07K 1/22

(52) U.S. Cl. CPC C07K 1/22

(57) ABSTRACT

A method for purifying a target protein from a cell culture sample containing the target protein, viral compounds and other impurities, by affinity chromatography virus inactivation and optionally other purifications the affinity chromatography involving

a) loading an affinity chromatography column with the cell culture sample thereby binding the target protein to the affinity chromatography column;
b) eluting the target protein from the affinity chromatography column by contacting the affinity chromatography column with an elution buffer having a pH<6 and comprising an excipient, wherein the excipient is disaccharides, polyols or poly (ethylene glycol) polymers;
c) collecting one or more fractions containing the target protein obtained from (b);
d) potentially combining the fractions obtained from (c) to form an elution product pool,
and wherein the virus inactivation involves
e) incubating the elution product pool at a pH from 2.5 to 4.5.

### TECHNICAL FIELD

The present invention relates to an improved method for purifying a target protein from a cell culture sample, wherein the cell culture sample comprises the target protein, viral compounds and other product and process related impurities, comprising an affinity chromatography step, a virus inactivation step and optionally other purification steps.

### STATE OF THE ART

The therapeutic applications for proteins and particularly monoclonal antibodies (mAbs) play an increasing role in today's medical needs.

Key aspects during the downstream processing of biotechnologically produced proteins are purity of the target protein and process yield. Therefore, the downstream process needs to be designed in a way that the final product which will eventually end up in a therapeutic agent that is administered to a patient. Therefore, it is important that the final therapeutic agent exhibits low levels of product and process related impurities (e.g. high molecular weight aggregates) as well as process related contaminants (e.g. host cell protein levels, DNA, endotoxin, leached Protein A and some cell culture media additives). In addition to this, the process has to be capable of clearing and inactivating viruses to ensure product safety.

Protein and particularly mAb purification is a complex and cost-intensive multi-step process which typically involves Protein A affinity chromatography. Protein A affinity chromatography is a highly selective mAb purification step starting from complex cell culture media and resulting in typically more than 95% mAb purity. The impurities such as media proteins, host cell protein, nucleic acids and endotoxin are removed in the flow-through when the mAb containing sample solution is passed through a protein A column, while the mAb product is retained within the column. The mAb product is eluted from protein A resins by lowering the pH by using acidic eluting buffer which reduces the interaction between mAb and protein A. The acidic condition after the elution step is also suitable for inactivation of pH sensitive viral contaminants (Yoo, S. M., Ghosh, R. 2012. Simultaneous removal of leached protein-A and aggregates from monoclonal antibody using hydrophobic interaction membrane chromatography. Journal of Membrane Science, 390: 263-269). Therefore, after elution the mAb product from Protein A chromatography is typically subjected to viral inactivation by incubation at low pH since the Protein A column elutes in a low pH buffer.

A limitation of Protein A chromatography and virus inactivation is the need to carry out the elution of a protein or antibody from the Protein A resin and the virus inactivation step under acidic conditions. Low pH treatment has been shown to successfully inactivate retroviruses for a variety of biotechnology products (Brorson, K., Krejci, S., Lee, K., Hamilton, E., Stein, K., Xu, Y. 2003. bracketed generic inactivation of rodent retroviruses by low pH treatment for monoclonal antibodies and recombinant proteins, Biotechnology and Bioengineering 82, 321-329). However, exposure to low pH conditions can result in the formation of soluble high molecular weight aggregates and/or insoluble precipitates during product elution. High molecular weight aggregate formation can lead to a reduction in product yield if a significant level of the product species aggregate.

Strategies to address protein aggregation during Protein A chromatography by adding excipients e.g. arginine and urea as protein stabilizer at low pH during Protein A chromatography have been described. Addition of urea was effective in reducing on-column and in-solution aggregation at concentration of 0.5M and 1M, respectively (Shukla, A. A., Hubbard, B., Tressel, T., Guhan, S., Low, D. 2007. Downstream processing of monoclonal antibodies-application of platform approaches. J Chromatogr B Analyt Technol Biomed Life Sci 848(1):28-39). Protein A chromatography using arginine solutions as eluent was found to prevent protein aggregation on elution from protein A (Arakawa, T., Philo, J. S., Tsumoto, K., Yumioka, R., Ejima, D. 2004. Elution of antibodies from a protein-A column by aqueous arginine solutions, Protein Expr. Purif. 36, 244-248).

There is still a need in the biopharmaceutical industry to define improved methods in order to reduce the risk of protein aggregation during low pH steps in downstream processing. Especially an approach towards adding pharmaceutically acceptable stabilizing excipients to the elution buffer in protein A affinity chromatography is of high interest, since this buffer system also plays a crucial role in the following critical processing step of viral inactivation.

### SUMMARY OF THE INVENTION

It was surprisingly found that in purification processing of biopharmaceutical proteins such as mAb, the addition of a neutral excipient selected from the group consisting of disaccharides, polyols and poly (ethylene glycol) polymers to the elution buffer in Protein A affinity chromatography prevents aggregation and precipitation of the target protein resulting in an enhanced product yield in the elution product pool. It was further found that the selected excipients effectively stabilize mAbs during low pH treatment in a viral inactivation step and do not interfere with the viral inactivation during the low pH treatment. As the selected excipients are acceptable and useful in pharmaceutical preparations comprising the target mAbs there is no need to remove the excipient in further processing steps.

In particular, the invention provides a method for purifying a target protein from a cell culture sample, wherein the cell culture sample comprises the target protein, viral compounds and other product and process related impurities, comprising an affinity chromatography step, a virus inactivation step and optionally other purification steps, wherein the affinity chromatography step comprises

a) loading an affinity chromatography column with the cell culture sample thereby binding the target protein to the affinity chromatography column;
b) eluting the target protein from the affinity chromatography column by contacting the affinity chromatography column with an elution buffer having a pH<6 and comprising an excipient, wherein the excipient is selected from the group consisting of disaccharides, polyols and poly (ethylene glycol) polymers;
c) collecting one or more fractions containing the target protein obtained from step (b);
d) combining the fractions obtained from step (c) to form an elution product pool;
and wherein the virus inactivation step comprises
e) incubating the elution product pool at a pH from 2 to 5.

According to a preferred embodiment of the invention, the affinity chromatography step is a Protein A affinity chromatography step.

According to another preferred embodiment of the invention, the target protein is a monoclonal antibody.

According to another preferred embodiment of the invention, the poly (ethylene glycol) polymer has an average molecular weight from 1,000 g/mol to 10,000 g/mol.

According to an advantageous aspect of the invention, the excipient is selected from the group consisting of sucrose, trehalose, sorbitol, mannitol and PEG4000.

In a preferred embodiment of the invention, the elution buffer has an excipient concentration from 2% to 15% by weight, still more preferred from 5% to 10% by weight.

In another preferred embodiment of the invention the elution buffer is a citrate buffer.

Preferably, the elution buffer has a pH from 2.5 to 5.5.

According to a further advantageous aspect of the invention, the elution step (b) comprises contacting the affinity chromatography column with the elution buffer using an elution buffer gradient from pH 5.5 to pH 2.75.

According to another advantageous aspect of the invention, the pH of the elution product pool is adjusted to a pH in the range from pH 2 to pH 5 prior to the incubation step (e).

According to another advantageous embodiment of the invention the incubation step (e) is performed at pH 2.5 to pH 4.5.

According to another preferred embodiment of the invention, the incubation step (e) is performed at room temperature.

### DETAILED DESCRIPTION OF THE INVENTION

In optimizing downstream processing of biopharmaceutical proteins the focus is on obtaining high product yields and high product purities. However, many biopharmaceutically active proteins and particularly monoclonal antibodies tend to form dimers, oligomers or higher order aggregates and precipitate during processing steps which are carried out at low pH conditions such as affinity chromatography steps and viral inactivation steps. In order to provide a therapeutic protein product with the required purity, these agglomerate protein species have to be removed during the purification process. The present invention now provides a method for purifying a target protein from a cell culture sample, wherein the cell culture sample comprises the target protein, viral compounds and other product and process related impurities, comprising an affinity chromatography step, a virus inactivation step and optionally other purification steps, wherein the affinity chromatography step comprises elution of the target protein with an elution buffer having a pH<6 and comprising an excipient selected from the group consisting of disaccharides, polyols and poly (ethylene glycol) polymers. The addition of one of the selected excipients to the elution buffer was found to stabilize the target protein in low pH solutions which is reflected in low protein aggregation and high yields of the target protein. It has surprisingly been found that the selected excipients do not interfere with a subsequent viral inactivation step which is also carried out at low pH conditions. Rather, it was found that the selected excipients also stabilize the target protein during low pH incubation periods. Since the selected excipients are pharmaceutically acceptable and may safely be administered to humans and animals, there is no need to remove them from the purification process. This allows optimizing downstream processing of biopharmaceutical proteins towards lower cost and reduced processing time.

The term “affinity chromatography” shall refer to chromatography processes of separating biochemical mixtures based on a highly specific interaction between e.g. antigen and antibody, enzyme and substrate, receptor and ligand, or protein and nucleic acid. Examples of such chromatographic resins include, but are not limited to Protein A resin, Protein G resin, Protein L resin, immobilized metal ion affinity chromatography etc.

In a particular embodiment of the invention, the affinity chromatography column is a Protein A affinity chromatography column.

The term “Protein A affinity chromatography” shall refer to the separation or purification of substances and/or particles using protein A, where the protein A is generally immobilized on a solid phase. Protein A is a 40-60 kD cell wall protein originally found in Staphylococcus aureus. The binding of antibodies to protein A resin is highly specific. Protein A affinity chromatography columns for use in protein A affinity chromatography herein include, but are not limited to, Protein A immobilized on a polyvinylether solid phase, e.g. the Eshmuno® columns (Merck, Darmstadt, Germany), Protein A immobilized on a pore glass matrix, e.g. the ProSep® columns (Merck, Darmstadt, Germany) Protein A immobilized on an agarose solid phase, for instance the MABSELECT™ SuRe™ columns (GE Healthcare, Uppsala, Sweden).

The present invention may include further purification steps that are commonly applied in purification processes of cell-culture derived target proteins. Non-limiting examples are column chromatography steps such as an affinity chromatography column, a hydrophobic interaction column and an ion exchange column as well as filtration steps such as ultrafiltration and diafiltration.

The term “cell culture sample” shall refer to a sample derived from cell culture media, i.e. a solution used during culturing, growth, or maintenance of a cell, particularly a mammalian host cell, and comprising a target protein of interest. As used herein, the cell culture sample comprising the target protein may be a harvested cell culture fluid sample or may be the eluate from a preceding filtration and/or chromatography step.

A “protein” is a macromolecule comprising one or more polypeptide chains or at least one polypeptide chain of more than 100 amino acid residues. A polypeptide may also comprise non-peptidic components, such as carbohydrate groups. Carbohydrate groups and other non-peptidic substituents may be added to a polypeptide by the cell in which the polypeptide is produced, and will vary with the type of cell. Polypeptides are defined herein in terms of their amino acid backbone structures; substituents such as carbohydrate groups are generally not specified, but may be present nonetheless.

As used herein, the term “antibody” refers to any form of antibody or fragment thereof and is a protein that exhibits a desired biological activity. Thus, it is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. “Isolated antibody” refers to the purification status of a binding compound and in such context means the molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

The term “monoclonal antibody” or “mAb”, as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. In contrast, conventional (polyclonal) antibody preparations typically include a multitude of antibodies directed against (or specific for) different epitopes. The modifier “monoclonal” indicates the character of the antibody as being obtained from a substantially homogeneous population of antibodies and is not to be construed as requiring production of the antibody by any particular method. For example, the monoclonal antibodies to be used in accordance with the present invention may be made by the hybridoma method first described by Kohler et al., (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al., (1991) Nature 352: 624-628 and Marks et al., (1991) J. Mol. Biol. 222: 581-597, for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., (1984) Proc. Natl. Acad. Sci. USA 81: 6851-6855).

In order to recover the target protein or antibody in monomeric form from the affinity chromatography column, the adsorption is followed by eluting the adsorbed protein in monomeric form from the affinity chromatography resin. Elution of adsorbed protein can be effected by applying an elution buffer changing the pH conditions of the mobile phase in the column as compared to the previous adsorption step.

The term “mobile phase” denotes any mixtures of water and/or aqueous buffer and/or organic solvents being suitable to recover polypeptides from a chromatography column. The term “to elute” or “eluting”, respectively, in the present context is used as known to the expert skilled in the art and denotes the dissolution, optionally the displacement, of adsorbed substance(s) from solids or adsorbents, which are impregnated with fluids, i.e., the column material to which the substance(s) is/are adsorbed.

The term “buffer” as used herein shall refer to a buffered solution that resists changes in pH by the action of its acid-base conjugate components. The “elution buffer” is the buffer which is used to elute a protein from the chromatography column. The elution buffer for the affinity chromatography step of this invention typically has a pH<6. A person skilled in the art is aware that the choice of pH largely depends on the stability profile of the target protein of interest. In a preferred embodiment, the pH is in a range from 2.5 to 5.5. Examples of buffers that will control the pH within this range include phosphate, acetate, citrate or ammonium buffers, or combinations thereof. The preferred such buffer is citrate.

In the present invention, the elution buffer comprises an excipient which is selected from the group consisting of disaccharides, polyols and poly (ethylene glycol).

In an embodiment of the invention, the excipient is a pharmaceutically acceptable compound. The term “pharmaceutically acceptable compound” refers to a compound which is non-toxic at the dosages and concentrations employed to the patent and that is compatible with other ingredients of the pharmaceutical formulation.

In one embodiment the excipient is a disaccharide. In a further embodiment the disaccharide is sucrose, or trehalose.

In another embodiment the excipient is a polyol. In a preferred embodiment, the polyol denotes a sugar alcohol that has at least four hydroxyl groups. Thus in one embodiment a polyol is selected from a tetrol that has four free hydroxyl groups, or a pentaol that has five free hydroxyl groups, or a hexaol that has six free hydroxyl groups. In a preferred embodiment the polyol is sorbitol or mannitol.

In one embodiment of the invention, the excipient is a poly (ethylene glycol) polymer. Although poly (ethylene glycol) polymers vary substantially by molecular weight, polymers having molecular weights ranges from about 400 g/mol to about 30,000 g/mol are usually suitable. In preferred embodiments of the invention, polyethylene glycols having an average molecular weight in the range of 1,000 g/mol to 10,000 g/mol, more preferred from 3,000 g/mol to 5,000 g/mol are suitably selected. In the examples of the invention, polyethylene glycol of an average molecular weight of 4,000 g/mol (PEG4000) was selected.

In a preferred embodiment, the elution buffer has an excipient concentration from 2% to 15% by weight, still more preferred from 5% to 10% by weight. Any excipient can be used at a concentration that is higher than the concentration necessary to achieve the intended stabilization effect. A person skilled in the art can determine the excipient concentration range in that the effect is present and that can be tolerated in the method as reported herein.

In one embodiment one or more excipients may be present in the elution buffer applied to the chromatography material for eluting the target protein, especially the antibody. In one embodiment, the elution buffer comprises up to five different excipients. If more than one excipient is present in the solution the sum of the concentrations of all excipients present in the solution is preferably within the range as defined above. For any single excipient or any combination of excipients a skilled person will consider the individual solubilities when determining the suitable concentration in the elution buffer.

In a preferred embodiment according to the process of the present invention, bind and elute chromatography step is followed by virus inactivation.

Preferably, the output or eluate from bind and elute chromatography (affinity chromatography step) is subjected to virus inactivation. Viral inactivation renders viruses inactive, or unable to infect, which is important, especially in case the target molecule is intended for therapeutic use.

Many viruses contain lipid or protein coats that can be inactivated by chemical alteration. Rather than simply rendering the virus inactive, some viral inactivation processes are able to denature the virus completely. Methods to inactivate viruses are well known to a person skilled in the art. Some of the more widely used virus inactivation processes include, e.g., use of one or more of the following: solvent/detergent inactivation (e.g. with Triton X 100); pasteurization (heating); acidic pH inactivation; and ultraviolet (UV) inactivation. It is possible to combine two or more of these processes; e.g., perform acidic pH inactivation at elevated temperature.

In order to ensure complete and effective virus inactivation, virus inactivation is often performed over an extended period of time with constant agitation to ensure proper mixing of a virus inactivation agent with the sample. For example, in many processes used in the industry today, an output or eluate from a capture step is collected in a pool tank and subjected to virus inactivation over an extended period of time (e.g., >1 to 2 hours, often followed by overnight storage).

In various embodiments described herein, the time required for virus inactivation can be significantly reduced by performing virus inactivation in-line or by employing a surge tank instead of a pool tank for this step. Examples of virus inactivation techniques that can be used in the process described herein can be found, e.g., in US2017320909 (A1), which is incorporated here by reference.

In a preferred embodiment of the present invention, virus inactivation employs use of acidic pH, where the output from the bind and elute chromatography step is subjected to exposure to acidic pH for virus inactivation, either using a surge tank or in-line. The pH used for virus inactivation is typically less than 5.0, or preferably between 3.0 and 4.0. In some embodiments, the pH is about 3.6 or lower. The duration of time used for virus inactivation using an in-line method can be anywhere between 10 minutes or less, 5 minutes or less, 3 minutes or less, 2 minutes or less, or about 1 minute or less. In case of a surge tank, the time required for inactivation is typically less than 1 hour, or preferably less than 30 minutes.

In some embodiments of the invention described herein, a suitable virus inactivation agent is introduced in-line between the chromatography process step and the next unit operation in the process (e.g., flow through purification). Preferably the tube or connecting line contains a static mixer which ensures proper mixing of the output from the chromatography process step with the virus inactivation agent, before the output goes on to the next unit operation. Typically, the output from the bind and elute chromatography flows through the tube at a certain flow rate, which ensures a minimum contact time with the virus inactivation agent. The contact time can be adjusted by using tubes of a certain length and/or diameter.

In some embodiments, a base or a suitable buffer is additionally introduced into the tube or connecting line after exposure to an acid for a duration of time, thereby to bring the pH of the sample to a suitable pH for the next step, where the pH is not detrimental to the target molecule. Accordingly, in a preferred embodiment, both exposure to a low pH as well as that to a basic buffer is achieved in-line with mixing via a static mixer.

In some embodiments, instead of an in-line static mixer, or in addition to an in-line static mixer, a surge tank is used for treating the output from the bind and elute chromatography step with a virus inactivation agent, where the volume of the surge tank is not more than 25% of the total volume of the output from the bind and elute chromatography step or not more than 15% or not more than 10% of volume of the output from the bind and elute chromatography step. Because when the volume of the surge tank is significantly less than the volume of a typical pool tank, more efficient mixing of the sample with the virus inactivation agent can be achieved.

In some embodiments, virus inactivation can be achieved by changing the pH of the elution buffer in the bind and elute chromatography step, rather than having to add acid to the output from the affinity chromatography step. Typically, following virus inactivation, the sample is subjected to a flow-through purification process.

In some embodiments, a filtration step may be included after virus inactivation and before flow through purification. Such a step may be desirable, especially in cases turbidity of the sample is observed following virus inactivation (i.e., after addition of both acid and base). In some embodiments, the filtration step may include a microporous filter or a depth filter.

As previously described, it has been found that the protein to be purified can be stabilized and turbidity and unwanted aggregation can be avoided by the addition of suitable excipients. Thus, In a preferred embodiment of the present invention both, the virus inactivation step and the affinity chromatography step are carried out in presence of at least an excipient, which is selected from the group consisting of disaccharides, polyols and poly (ethylene glycol) polymers. In a more preferred embodiment the added excipient is selected from the group consisting of sucrose, trehalose, sorbitol, mannitol and PEG4000.

In this way, the desired protein can be obtained in purified and stabilized form, while maintaining virus inactivation.

In the present invention, the elution product pool obtained from the affinity chromatography step is exposed to pH viral inactivation. Exposure to acidic pH reduces or completely eliminates pH sensitive viral contaminants. The pH viral inactivation step comprises incubating the elution product pool at a pH from 2 to 5, preferably from 2.5 to 4.5, particularly preferred from 2.8 to 3.6 for a period of time. Typically, the pH viral inactivation step is finished by neutralizing the pH and, where necessary, removing particulates by filtration.

In another embodiment of the invention, the pH of the elution product pool may be adjusted to the pH desired for the viral inactivation step. In one embodiment, the pH of the elution product pool has to be lowered by adding an acid including, but not limited to, citric acid, acetic acid, caprylic acid, or other suitable acids. The choice of pH level depends on the stability profile of the target protein components. According to the present invention, the applied excipient which is present in the elution product pool may enhance the stability of the target protein during low pH viral inactivation.

The stability of the target protein during low pH virus inactivation is also affected by the duration of the low pH incubation. In one embodiment, the duration of the low pH incubation is from 30 min to 120 min, preferably from 30 min to 60 min.

In another embodiment, the virus inactivation performed at room temperature.

### BRIEF DESCRIPTION OF THE FIGURES

FIG. 1 shows the stabilizing effect of certain excipients on mAbA during low pH treatment. The upper curves with triangle markers shows the stabilizing effect of an exemplary neutral excipient (0.5M sorbitol) on mAbA during low pH treatment indicated by a stable or increasing mAbA monomer content over the incubation time at pH 2.8 measured by kinetic SEC. In comparison as a negative control, the lower curves with circle markers shows a destabilizing effect of an exemplary ionic excipient (0.5M arginine HCl) at low pH conditions which is indicated by a significant decrease in mAbA monomer content over the incubation time at pH 2.8 (Example 1).

FIG. 2 is a bar diagram showing effects of certain excipients (sorbitol and arginine HCl) during low pH treatment measured by nanoDSF (Example 1.5). Higher Tm-values than “no additive control” (e.g. for 0.5M sorbitol) indicate stabilizing properties. A destabilizing effect was observed for the addition of arginine HCl.

FIG. 3 is a bar diagram showing summarized effects of selected excipients (sorbitol, mannitol, sucrose, trehalose, PEG4000 and arginine HCl) on mAbA stability during low pH treatment. Based on the results of kinetic SE-HPLC and nanoDSF, the selected neutral excipients (sorbitol, mannitol, sucrose, trehalose and PEG4000) showed a stabilizing effect during the stress condition as indicated by a positive delta value. PEG4000 however could only stabilize the mAbA in the citrate buffer system without the addition of NaCl (Example 1).

FIG. 4 is a bar diagram showing summarized effects of selected excipients (sorbitol, mannitol, sucrose, trehalose, PEG4000 and arginine HCl) on mAbB stability during low pH treatment. Based on the results of kinetic SE-HPLC and nanoDSF, the selected neutral excipients (sorbitol, mannitol, sucrose, trehalose and PEG4000) showed a stabilizing effect during the stress condition as indicated by the decrease in increase of monomer and Tm-values. PEG4000 however could only stabilize the mAbB in the citrate buffer system without the addition of NaCl (Example 1).

FIG. 5 is a bar diagram showing improved stability of mAbA caused by selected neutral excipients (sucrose, mannitol, trehalose, and PEG4000 and sorbitol) during low pH virus inactivation at pH 2.8 for 60 minutes (Example 4).

FIG. 6 is a bar diagram showing improved stability of mAbB caused by selected neutral excipients (sucrose, mannitol, trehalose, and PEG4000 and sorbitol) during low pH virus inactivation at pH 2.8 for 60 minutes (Example 4).

FIG. 7 is a flow diagram showing the process steps for low pH treatment at pH 3.6 using MLV virus (Example 5).

FIG. 8 is a diagram showing viral reduction factors for MLV in the presence of selected neutral excipients (sorbitol, mannitol, sucrose, trehalose and PEG4000) against the incubation time during low pH treatment (Example 6).

FIG. 9 is a bar diagram showing viral reduction factors for MLV virus in the presence of selected neutral excipients (sorbitol, mannitol, sucrose, trehalose and PEG4000) after 60 min of low pH treatment (Example 6).

### EXAMPLES

### Example 1: Stabilizing Effect of Selected Excipients on Low pH Induced Aggregation Test (In-Vitro)

The effect of the use of neutral excipients on mAbs during low pH stress conditions simulating the protein A chromatography and virus inactivation steps during downstream processing of monoclonal antibodies have been evaluated in-vitro. The in-vitro screening tests have been performed with an incubation experiment of two model proteins (mAbA and mAbB) at low pH values with or without the addition of NaCl. The effects of these experiments on the conformational stability, fragmentation and aggregation behavior of the samples were analyzed using kinetic-SEC and nanoDSF and compared against control conditions without exipient.

Furthermore, an ionic excipient (arginine HCl) was also used as a negative control to show the destabilizing effect of unsuitable excipients for incubation at low pH condition.

### Example 1.1: Preparation of 0.25M Citrate Buffer pH 3.0

Solution A: 0.25M Citric Acid Monohydrate (C₆H₈O₇.H₂O FW=210.14)

52.5 g citric acid monohydrate (M=210.14 g/mol) was weighed into an appropriate flask. 500 ml milli-Q-water was added and the solution was stirred until the substance was completely dissolved.

Solution B: 0.25M Trisodium Citrate, Dihydrate (C₆H₅O₇Na₃.2H₂O FW=294.12)

18.4 g trisodium citrate, dihydrate (M=294.12 g/mol) was weighed into an appropriate flask. 500 ml milli-Q-water was added and the solution was stirred until the substance was completely dissolved.

Approximately 415 ml of solution A and approximately 85 ml of Solution B were mixed too obtain approximately 500 ml 0.25M citrate buffer pH 3.0. The pH was adjusted to 3.0±0.05 using 1M HCl solution or 1M NaOH if necessary.

The buffer was filtered using a 0.45 μm HAWP mixed cellulose ester filter (Merck, Darmstadt, Germany) and degassed for 20 min in an ultrasonic bath before use.

### Example 1.2: Protein Sample Preparation

The tested proteins are mAbA and mAbB.

mAbA is a monoclonal antibody (app. 152 kDa) with a pl˜7.01-8.58. It was post TFF purified mAb and formulated with 10 mM citrate buffer pH 5.5, 0.1M NaCl, 0.1M Glycine. The solution has a concentration of 16 mg/mL.

mAbB is a monoclonal antibody (app. 145 kDa) with a pl˜7.6-8.3. It was post TFF purified mAb and formulated with 50 mM sodium acetate pH 5.0. The solution has a concentration of 80 mg/mL.

TABLE 1
Sample preparation for in vitro excipient screening

| | Final Screening Conditions | | | | | | | | | | | | | |
|---|---|---|---|---|---|---|---|---|---|---|---|---|---|---|
| | 0.1M citrate buffer pH 2.8 | | | | | | | | | | | | | |
| | no | | 5% | | 0.5M | | 0.5M | | 0.5M | | 0.5M | | 0.5M | |
| | excipient | | PEG4000 | | sucrose | | trehalose | | sorbitol | | mannitol | | arginine HCl | |
| Stock | or | | or | | or | | or | | or | | or | | or | |
| Solution | (+0.05M NaCl) | | (+0.05M NaCl) | | (+0.05M NaCl) | | (+0.05M NaCl) | | (+0.05M NaCl) | | (+0.05M NaCl) | | (+0.05M NaCl) | |
| 0.25M citrate | 20000 | μl | 20000 | μl | 20000 | μl | 20000 | μl | 20000 | μl | 20000 | μl | 20000 | μl |
| buffer pH 3.0 | | | | | | | | | | | | | | |
| 12.5% | 0 | μl | 20000 | μl | 20000 | μl | 20000 | μl | 20000 | μl | 20000 | μl | 20000 | μl |
| PEG4000 or | | | | | | | | | | | | | | |
| 1.25M polyols | | | | | | | | | | | | | | |
| and | | | | | | | | | | | | | | |
| Disaccharides | | | | | | | | | | | | | | |
| or arginine-HCl | | | | | | | | | | | | | | |
| 5M NaCl | 0 | μl | 0 | μl | 0 | μl | 0 | μl | 0 | μl | 0 | μl | 0 | μl |

| | | | | | | |
|---|---|---|---|---|---|---|
| (or 500 μl) | (or 500 μl) | (or 500 μl) | (or 500 μl) | (or 500 μl) | (or 500 μl) | (or 500 μl) |

pH setting to 2.8 using 1M HCl
Milli-Q Water ad 50 ml

### Example 1.3: Stress Condition

The stress conditions were initiated by diluting the mAb-samples 1:20 (final conc. 0.8 mg/ml for mAbA and 4 mg/ml for mAbB) with the selected buffer condition (0.1M citrate buffer pH 2.8). The first samples were directly measured in SE-HPLC after dilution with selected buffers. The aggregation kinetics were monitored by repeating the measurement every 30 minutes for 2 hours. All samples were also measured by nano Differential Scanning Fluorimetry (nanoDSF) for the melting point (Tm) analysis. The different excipient formulations were prepared with these stock solution (pipetting scheme of buffer condition see Table 1).

### Example 1.4: Size Exclusion Chromatography (SEC) Conditions

Column: TSKgel SuperSW3000
System: Agilent 1290 UHPLC
Flow: 0.35 ml/min
Eluent: 0.025M NaH₂PO₄*H₂O/0.025M Na₂HPO₄/0.4M NaClO₄*H₂O/pH 6.3
Sample: mAbA and mAbB in low pH screening conditions

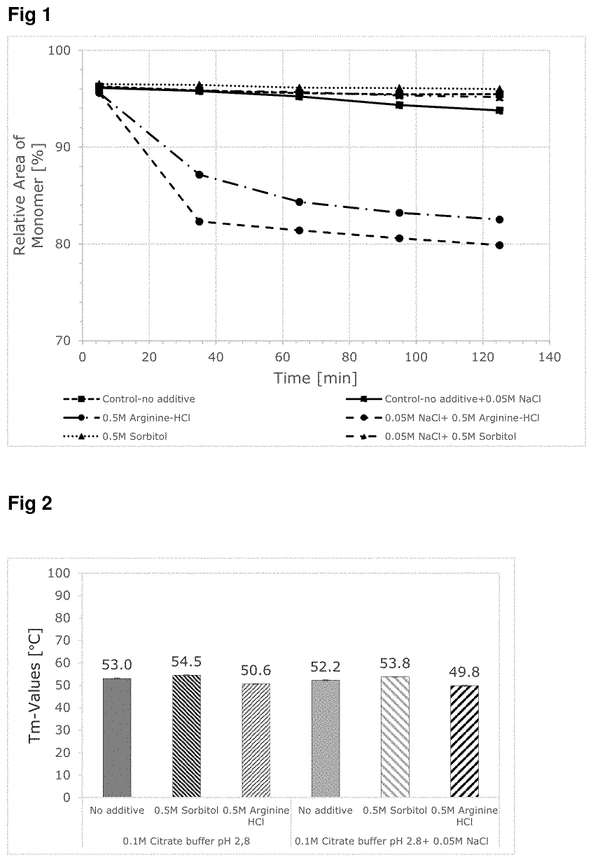

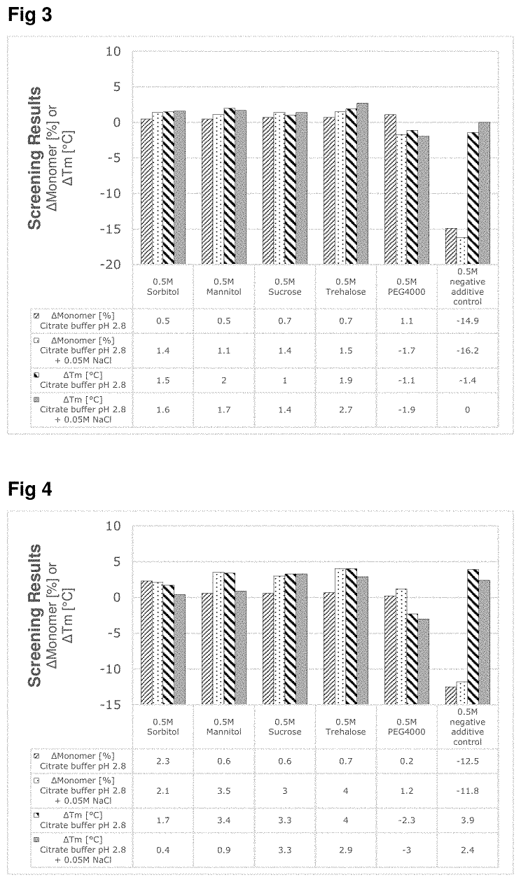

The results concerning the protein stabilizing effect of the excipients sorbitol and arginine HCl are shown in FIG. 1. A protein stabilizing effect was observed for the addition of 0.5M sorbitol; destabilizing effect was observed for the addition of 0.5M arginine HCl.

### Example 1.5: NanoDSF Conditions

NanoDSF is a modified differential scanning fluorimetry method to determine protein stability employing intrinsic tryptophan or tyrosin fluorescence. Protein stability can be addressed by thermal unfolding experiments. The thermal stability of a protein is typically described by the “melting temperature” or “Tm”, at which 50% of the protein population is unfolded, corresponding to the midpoint of the transition from folded to unfolded.

Analysis was performed with a Prometheus NT 48 (NanoTemper Technologies GmbH, Munich, Germany). The sample volume was 10 μl and the heating rate 1° C./min. whereas the temperature ramp started at 20° C. and lasts till 95° C.

The results concerning the protein stabilizing effect of the excipients sorbitol and arginine HCl are shown in FIG. 2. A protein stabilizing effect was observed for the addition of 0.5M sorbitol; destabilizing effect was observed for the addition of 0.5M arginine HCl.

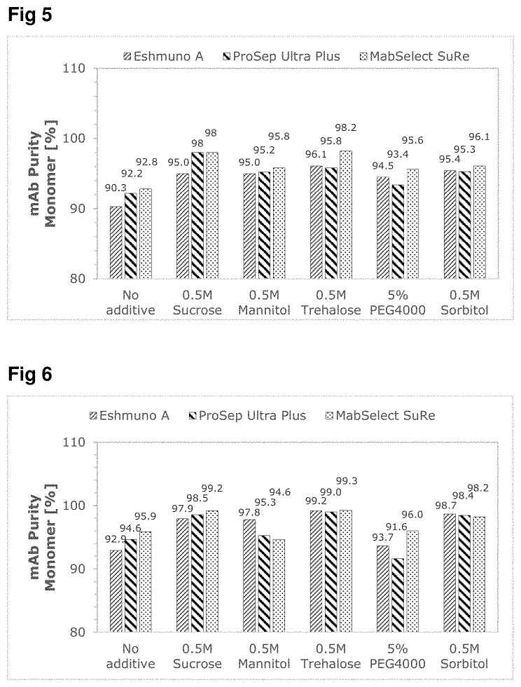

Based on the screening results with selected excipients (sorbitol, mannitol, sucrose, trehalose, PEG4000 and arginine HCl) as shown in FIG. 4 and FIG. 5 it was found that neutral excipients such as polyols (e.g. mannitol, Sorbitol) and disaccharides (e.g. sucrose, trehalose) as well as PEG4000 effectively stabilize mAbs in solution during low pH treatment.

### Example 2: Preparation of Buffer and Excipient Solutions for Protein A Chromatography

All buffers and excipients were filtered using a 0.45 μm HAWP mixed cellulose ester filter (Merck, Darmstadt, Germany) and degassed for 20 min in an ultrasonic bath before use. For all Protein A chromatography runs the following buffers were prepared and used:

TABLE 2
Buffer A1 for Protein A chromatography pH 5.50
Buffer A1, pH 5.50

| Substance | Concentration [mmol/L] | Concentration [g/L] |
|---|---|---|
| Citric acid*1H₂O | 100.0 | 21.01 |
| NaOH | 255.0 | Titrated until pH 5.50 |

TABLE 3
Buffer A2 for Protein A chromatography pH 7.00
Buffer A2, pH 7.00

| Substance | Concentration [mmol/L] | Concentration [g/L] |
|---|---|---|
| Citric acid*1H₂O | 100.0 | 21.01 |
| NaOH | 298.0 | Titrated until pH 7.00 |

TABLE 4
Buffer B for Protein A chromatography pH 2.75
Buffer B, pH 2.75

| Substance | Concentration [mmol/L] | Concentration [g/L] |
|---|---|---|
| Citric acid*1H₂O | 100.0 | 21.01 |
| NaOH | 33.0 | Titrated until pH 2.75 |

The following excipients were chosen based on their ability to protect antibodies from aggregation:

TABLE 5
Applied excipients with applied concentrations,
manufacturer and quality standard

| Substance | Applied concentrations | Manufacturer/Quality |
|---|---|---|
| Sucrose | 0.5M | Merck/Emprove ® |
| Sorbitol | 0.5M | Merck/Emprove ® |
| Mannitol | 0.5M | Merck/Emprove ® |
| Trehalose | 0.5M | Merck/For Biochemistry |
| PEG4000 | 5% (w/w) | Merck/Emprove ® |

### Example 2.1: Preparation of 0.5M Sucrose in Citrate Buffer pH 5.5

171.1 g sucrose (M=342.29 g/mol) was weighed into an appropriate flask. Approximately 800 ml 0.1M Na-citrate buffer pH 5.5 was added and the solution was stirred until the substance was completely dissolved. The pH was adjusted to 5.5+/−0.05 using 1M HCl. Afterwards, the solution was transferred to a 1000.0 ml volumetric graduated flask and filled to the mark with 0.1M Na-citrate buffer pH 5.5 and mixed thoroughly.

### Example 2.2: Preparation of 0.5M Sucrose in Citrate Buffer pH 2.75

171.1 g sucrose (M=342.29 g/mol) was weighed into an appropriate flask. Approximately 800 ml 0.1M Na-citrate buffer pH 2.75 was added and the solution was stirred until the substance was completely dissolved. The pH was adjusted to 2.75+/−0.05 using 1M HCl. Afterwards, the solution was transferred to a 1000.0 ml volumetric graduated flask and filled to the mark with 0.1M Na-citrate buffer pH 2.75 and mixed thoroughly.

### Example 2.3: Preparation of 0.5M Trehalose in Citrate Buffer pH 5.5

171.1 g trehalose (M=342.29 g/mol) was weighed into an appropriate flask. Approximately 800 ml 0.1M Na-citrate buffer pH 5.5 was added and the solution was stirred until the substance was completely dissolved. The pH was adjusted to 5.5+/−0.05 using 1M HCl. Afterwards, the solution was transferred to a 1000.0 ml volumetric graduated flask and filled to the mark with 0.1M Na-citrate buffer pH 5.5 and mixed thoroughly.

### Example 2.4: Preparation of 0.5M Trehalose in Citrate Buffer pH 2.75

171.1 g trehalose (M=342.29 g/mol) was weighed into an appropriate flask. Approximately 800 ml 0.1M Na-citrate buffer pH 2.75 was added and the solution was stirred until the substance was completely dissolved. The pH was adjusted to 2.75+/−0.05 using 1M HCl. Afterwards, the solution was transferred to a 1000.0 ml volumetric graduated flask and filled to the mark with 0.1M Na-citrate buffer pH 2.75 and mixed thoroughly.

### Example 2.5: Preparation of 0.5M Mannitol in Citrate Buffer pH 5.5

91.09 g mannitol (M=182.17 g/mol) was weighed into an appropriate flask. Approximately 800 ml 0.1M Na-citrate buffer pH 5.5 was added and the solution was stirred until the substance was completely dissolved. The pH was adjusted to 5.5+/−0.05 using 1M HCl. Afterwards, the solution was transferred to a 1000.0 ml volumetric graduated flask and filled to the mark with 0.1M Na-citrate buffer pH 5.5 and mixed thoroughly.

### Example 2.6: Preparation of 0.5M Mannitol in Citrate Buffer pH 2.75

91.09 g mannitol (M=182.17 g/mol) was weighed into an appropriate flask. Approximately 800 ml 0.1M Na-citrate buffer pH 2.75 was added and the solution was stirred until the substance was completely dissolved. The pH was adjusted to 2.75+/−0.05 using 1M HCl. Afterwards, the solution was transferred to a 1000.0 ml volumetric graduated flask and filled to the mark with 0.1M Na-citrate buffer pH 2.75 and mixed thoroughly.

### Example 2.7: Preparation of 0.5M Sorbitol in Citrate Buffer pH 5.5

91.09 g sorbitol (M=182.17 g/mol) was weighed into an appropriate flask. Approximately 800 ml 0.1M Na-citrate buffer pH 5.5 was added and the solution was stirred until the substance was completely dissolved. The pH was adjusted to 5.5+/−0.05 using 1M HCl. Afterwards, the solution was transferred to a 1000.0 ml volumetric graduated flask and filled to the mark with 0.1M Na-citrate buffer pH 5.5 and mixed thoroughly.

### Example 2.8: Preparation of 0.5M Sorbitol in Citrate Buffer pH 2.75

91.09 g sorbitol (M=182.17 g/mol) was weighed into an appropriate flask. Approximately 800 ml 0.1M Na-citrate buffer pH 2.75 was added and the solution was stirred until the substance was completely dissolved. The pH was adjusted to 2.75+/−0.05 using 1M HCl. Afterwards, the solution was transferred to a 1000.0 ml volumetric graduated flask and filled to the mark with 0.1M Na-citrate buffer pH 2.75 and mixed thoroughly.

### Example 2.9: Preparation of 5% (w/v) PEG4000 in Citrate Buffer pH 5.5

50 g PEG4000 (M=3500-4500 g/mol) was weighed into an appropriate flask. Approximately 800 ml 0.1M Na-citrate buffer pH 5.5 was added and the solution was stirred until the substance was completely dissolved. The pH was adjusted to 5.5+/−0.05 using 1M HCl. Afterwards, the solution was transferred to a 1000.0 ml volumetric graduated flask and filled to the mark with 0.1M Na-citrate buffer pH 5.5 and mixed thoroughly.

### Example 2.10: Preparation of 5% (w/v) PEG4000 in Citrate Buffer pH 2.75

50 g PEG4000 (M=3500-4500 g/mol) was weighed into an appropriate flask. Approximately 800 ml 0.1M Na-citrate buffer pH 2.75 was added and the solution was stirred until the substance was completely dissolved. The pH was adjusted to 2.75+/−0.05 using 1M HCl. Afterwards, the solution was transferred to a 1000.0 ml volumetric graduated flask and filled to the mark with 0.1M Na-citrate buffer pH 2.75 and mixed thoroughly.

### Example 3: Protein a Chromatography

### Example 3.1: Protein a Chromatography Resins

The Eshmuno® base material is a rigid and hydrophilic polymer based on polyvinylether. Immobilized onto it is the C domain of Staphylococcus aureus Protein A in a pentameric form, which is recombinantly produced in E. coli. Eshmuno® A is from Merck (Darmstadt, Germany) and the column was packed by Repligen GmbH (Ravensburg, Germany).

TABLE 6
Column parameters for
applied Eshmuno ® A resin

| | | |
|---|---|---|
| Column length | 2 | cm |
| Column inner diameter | 0.8 | cm |
| Column volume | 1 | mL |
| Mean particle diameter | ~50 | μm |

| | |
|---|---|
| Base material | hydrophilic polyvinylether |
| Functional group | recombinant Protein A |
| | produced in E. coli, |
| | derived from C domain of |
| | native Protein A |
| Lot # | K93457960 |
| Serial # | 00168 |

ProSep® Ultra Plus resin has a controlled pore glass matrix and recombinant native Protein A as a ligand bound to it. ProSep® Ultra Plus is from Merck (Darmstadt, Germany) and the column was packed by Repligen GmbH (Ravensburg, Germany).

TABLE 7
Column parameters for applied
ProSep ® Ultra Plus resin

| | | |
|---|---|---|
| Column length | 2 | cm |
| Column inner diameter | 0.8 | cm |
| Column volume | 1 | mL |
| Mean particle diameter | 60 | μm |

| | |
|---|---|
| Base material | controlled pore glass |
| Functional group | rec. native Protein A |
| Lot # | A4SA045AQ |
| Serial # | 00227 |

The MabSelect™ SuRe™ resin has an agarose matrix. Immobilized onto it through thio-ether is a recombinantly produced (E. coli) tetramer of an engineered Protein A domain with a C-terminal cysteine. This resin was produced by GE Healthcare (Uppsala, Sweden) and the column was packed by Repligen GmbH (Ravensburg, Germany).

TABLE 8
Column parameters for applied
MabSelect ™ SuRe ™ resin

| | | |
|---|---|---|
| Column length | 2 | cm |
| Column inner diameter | 0.8 | cm |
| Column volume | 1 | mL |
| Mean particle diameter | 85 | μm |

| | |
|---|---|
| Base material | rigid, highly cross- |
| | linked agarose |
| Functional group | alkali-stabilized protein |
| | A-derived domain |
| Serial # | 00620 |

### Example 3.2: Protein Sample Preparation

The first model protein is a monoclonal antibody mAbA (approximately 152 kDa) with a pl˜7.01-8.58. It was used as clarified cell culture harvest, which was filtrated using a VacuCap® 90 PF Filter Unit with 0.8/0.2 μm Supor® membrane (Pall Corporation, NY, USA). The solution has a concentration of 0.943 mg/mL, a pH of 7.0 and a conductivity of 12 mS/cm.

The second model protein is a monoclonal antibody mAbB (approximately 145 kDa) produced by Merck (Darmstadt, Germany), with a pl˜7.6-8.3. It was used as clarified cell culture harvest, which was filtrated using a VacuCap® 90 PF Filter Unit with 0.8/0.2 μm Supor® membrane (Pall Corporation, NY, USA). The solution has a concentration of 1.45 mg/mL, a pH of 7.0 and a conductivity of 12.87 mS/cm.

### Example 3.3: Protein a Chromatography Method

The Protein A chromatography was done using the following method parameters:

TABLE 9
Method parameters for Protein A chromatography

| Column | Eshmuno ® A, ProSep® Ultra Plus, | |
|---|---|---|
| | MabSelect ™ SuRe ™ | |
| System | Äkta purifier | |
| Flow | 120 | cm/h |
| | (1 mL/min) | |
| Sample | mAbA and mAbB HCCF | |
| Sample load | 30 | mg/mL |
| Fractionation | 2 | mL |
| Buffers | 0.1M Citric acid, pH 7; 0.1M Citric | |
| | acid, pH 5.5 and pH 2.75 with added | |
| | excipients (sucrose, trehalose, | |
| | mannitol, sorbitol or PEG4000) as | |
| | described in Example 1 | |
| Equilibration | 10 | CV |
| Inject | 20.69 | CV |
| Wash pH 7 | 5 | CV |
| Wash pH 5.5 | 10 | CV |
| | (pooled as 2 × 5 CV) | |
| Gradient | 30 | CV |
| 100% B | 5 | CV |

Elution was carried out at a defined gradient slope by applying a linear gradient of 30CV from pH 5.5 to pH 2.75.

### Example 4: Size Exclusion Chromatography

Following elution, the mAb containing elution product pool from the protein A chromatography was subjected to viral inactivation by holding the solution at low pH for 1 h at room temperature, followed by neutralization to the desired pH in a range between 4.0-8.0. The low pH treatment which mimics the viral inactivation process step, was initiated by adjusting the pH of the elution product pool to pH 2.8±0.05 by titration with 1M HCl. The effect of the low pH incubation on the different model proteins with or without added stabilizing excipients was subsequently analyzed by High Performance-Size Exclusion Chromatography (HP-SEC).

### Condition for HP-SEC Analysis:

| | |
|---|---|
| Column: | Tosoh TSKgel Super SW mAb HTP |
| System: | Äkta Micro |
| Flow: | 0.4 mL/min |
| Eluent: | 0.1M NaH2PO4/0.3M NaCl/0.02% NaN3/pH 7.00 |
| Sample: | fractions from the protein A chromatography column, |
| | different contents of mAbs |

FIG. 5 and FIG. 6 show the results of HP-SEC analysis. The high content of monomeric mAbs in the samples containing selected neutral excipients (sorbitol, mannitol, sucrose, trehalose and PEG4000) shows that these excipients have an overall positive effect on protein stability during Protein A chromatography and subsequent low pH virus inactivation steps.

### Example 5: Preparation of Buffer and Excipient Solutions for Virus Inactivation Experiments

### Example 5.1: Preparation of 1M Citric Acid Solution

21.01 g citric acid monohydrate (M=210.14 g/mol) were weighed into an appropriate flask. 100 ml milli-Q-water was added and the solution was stirred until the substance was completely dissolved. This solution was filtered using a 0.2 μm filter.

### Example 5.2: Preparation of 0.1M Citrate Buffer, pH 3.5

Solution A: 0.1M Citric Acid Monohydrate (C₆H₈O₇.H₂O FW=210.14)

21.01 g citric acid monohydrate (M=210.14 g/mol) were weighed into an appropriate flask. 1000 ml milli-Q-water was added and the solution was stirred until the substance was completely dissolved.

Solution B: 0.1M Trisodium Citrate, Dihydrate (C₆H₅O₇Na₃.2H₂O FW=294.12)

29.41 g trisodium citrate, dihydrate (M=294.12 g/mol) were weighed into an appropriate flask. 1000 ml milli-Q-water was added and the solution was stirred until the substance was completely dissolved.

Approximately 700 ml of solution A and approximately 300 ml of solution B were mixed to obtain approximately 1000 ml of 0.1M citrate buffer pH 3.5. The pH of the solution was adjusted to 3.5±0.05 using 1M citric acid solution or 1M NaOH if necessary.

### Example 5.3: Preparation of 0.5M Sorbitol in 0.1M Citrate Buffer pH 3.5

9.1 g sorbitol (M=182.17 g/mol) was weighed into an appropriate flask. Approximately 80 ml 0.1M citrate buffer pH 3.5 was added and the solution was stirred until the substance was completely dissolved. The pH was adjusted to 3.5±0.05 using 1M citric acid solution or 1M NaOH. Afterwards, the solution was transferred to a 100.0 ml volumetric graduated flask and filled to the mark with 0.1M citrate buffer pH 3.5 and mixed thoroughly. This solution was filtered using a 0.2 μm filter.

### Example 5.4: Preparation of 0.5M Mannitol in 0.1 M Citrate Buffer pH 3.5

9.1 g mannitol (M=182.17 g/mol) was weighed into an appropriate flask. Approximately 80 ml 0.1 M citrate buffer pH 3.5 was added and the solution was stirred until the substance was completely dissolved. The pH was adjusted to 3.5±0.05 using 1M citric acid solution or 1M NaOH. Afterwards, the solution was transferred to a 100.0 ml volumetric graduated flask and filled to the mark with 0.1 M citrate buffer pH 3.5 and mixed thoroughly. This solution was filtered using a 0.2 μm filter.

### Example 5.5: Preparation of 0.5M Sucrose in 0.1 M Citrate Buffer pH 3.5

17.1 g sucrose (M=342.29 g/mol) was weighed into an appropriate flask. Approximately 80 ml 0.1 M citrate buffer pH 3.5 was added and the solution was stirred until the substance was completely dissolved. The pH was adjusted to 3.5±0.05 using 1M citric acid solution or 1M NaOH. Afterwards, the solution was transferred to a 100.0 ml volumetric graduated flask and filled to the mark with 0.1 M citrate buffer pH 3.5 and mixed thoroughly. This solution was filtered using a 0.2 μm filter.

### Example 5.6: Preparation of 0.5M Trehalose in 0.1 M Citrate Buffer pH 3.5

17.1 g trehalose (M=342.29 g/mol) was weighed into an appropriate flask. Approximately 80 ml 0.1 M citrate buffer pH 3.5 was added and the solution was stirred until the substance was completely dissolved. The pH was adjusted to 3.5±0.05 using 1M citric acid solution or 1M NaOH. Afterwards, the solution was transferred to a 100.0 ml volumetric graduated flask and filled to the mark with 0.1 M citrate buffer pH 3.5 and mixed thoroughly. This solution was filtered using a 0.2 μm filter.

### Example 5.7: Preparation of 0.5M PEG4000 in 0.1 M Citrate Buffer pH 3.5

5 g PEG4000 (M=3500-4500 g/mol) was weighed into an appropriate flask. Approximately 80 ml 0.1 M citrate buffer pH 3.5 was added and the solution was stirred until the substance was completely dissolved. The pH was adjusted to 3.5±0.05 using 1M citric acid solution or 1M NaOH. Afterwards, the solution was transferred to a 100.0 ml volumetric graduated flask and filled to the mark with 0.1M citrate buffer pH 3.5 and mixed thoroughly. This solution was filtered using a 0.2 μm filter.

### Example 6: Effectiveness of Excipient on Viral Reduction During Low pH Inactivation Hold

Xenotropic murine leukaemia virus (MLV) was used as the model virus for the viral reduction experiments. MLV represents a non-defective gamma retrovirus. Inclusion of MLV is mandatory for biological products derived from CHO cell lines and monoclonal antibody products.

TABLE 10
Applied model virus

| | |
|---|---|
| Virus | MLV |
| Strain | pNFS Th-1 |
| Genome | ssRNA |
| Envelope | Yes |
| Family | Retro |
| Size (nm) | 80-110 |
| Resistance to physical/ | Low |
| chemical reagents | |

The applied model protein was mAbB as described in Example 1.2.

TABLE 11
Applied model protein

| Sample | Concentration [mg/ml] |
|---|---|
| mAbB | 130 |
| (IgG1, Mw: 145 kDa) | |

All assays were performed using the TCID₅₀ infectivity method.

### Starting Material Preparation

Prior to spiking, materials were thawed in a water bath at 37° C.±1° C., with gentle inversion and the container was removed from the water bath as soon as the ice has completely melted.

For the low pH load sample, the sample was received at 130 mg/ml and was diluted to a final concentration of 10 mg/ml using either buffer alone (0.1M citrate buffer pH 3.5 according to Example 5.2) or excipient in citrate buffer (according to Examples 4.3 to 4.7).

To adjust to the desired protein concentration, it was necessary to prepare in 1 in 13 dilution, e.g. add 1 part low pH load to 12 parts buffer or excipient in citrate buffer.

The samples were mixed throughout the manipulations and low pH hold. Once the sample temperature had reached 20° C.±0.5° C. the pH was adjusted to pH 3.6 using 1M citric acid and/or 1M Tris.

For spiking, 50 ml of pH adjusted sample was spiked. The remainder was adjusted to pH 6.0 to pH 8.0 with 1M Tris and 5 ml sample were dispensed for spiking. This sample was the time zero neutralized control and was spiked with 5% (v/v) MLV.

The virus spike (5% v/v) was added to the neutralized control sample. Then the sample was split equally to create the neutralized load sample and the load hold sample. The pH of the neutralized load sample was confirmed and the sample was placed on ice prior to titration. The load hold sample was retained at the same temperature as the bulk sample.

The virus spike of approximately 5% (v/v) was added to the pH adjusted 50 ml sample. 5 minutes after spiking (T=5 minutes), a sample was removed and immediately neutralized with 1M Tris.

Low pH treatment was performed at pH 3.6 to 3.64 (target pH 3.6) at 20° C.±0.5° C. The pH was monitored throughout the incubation period and adjusted if required to the target pH (pH 3.6).

Samples were removed at T=15 minutes and T=30 minutes. The pH of the samples was immediately adjusted to pH 6.0 to pH 8.0 with 1M Tris. After 60 minutes at pH 3.6, the remainder was adjusted to pH 6.0 to pH 8.0 with 1M Tris.

### Process Step

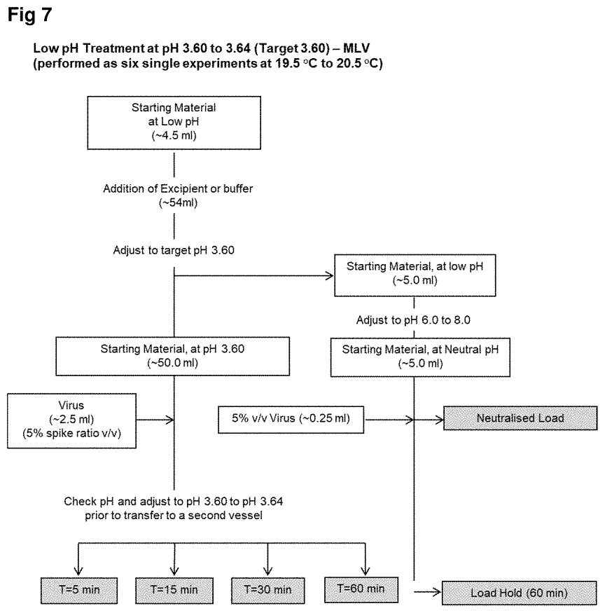

A chart recorder was used to monitor the temperature throughout each experiment. The interval of recording was every 1 minute. All process steps were as shown in FIG. 7 and described below and all volumes referenced in FIG. 7 are approximate volumes.

Following addition of the virus spike, the material was mixed thoroughly prior to any additional manipulations and collection of any samples.

Upon collection, all samples were mixed thoroughly and immediately neutralized int the range of pH 6.00 to 8.00 using 1M Tris when required. The required volume was placed on ice for assay and filtered using a 0.45 μm filter immediately prior to titration. A 0.45 μm filtered and an unfiltered positive control were inoculated.

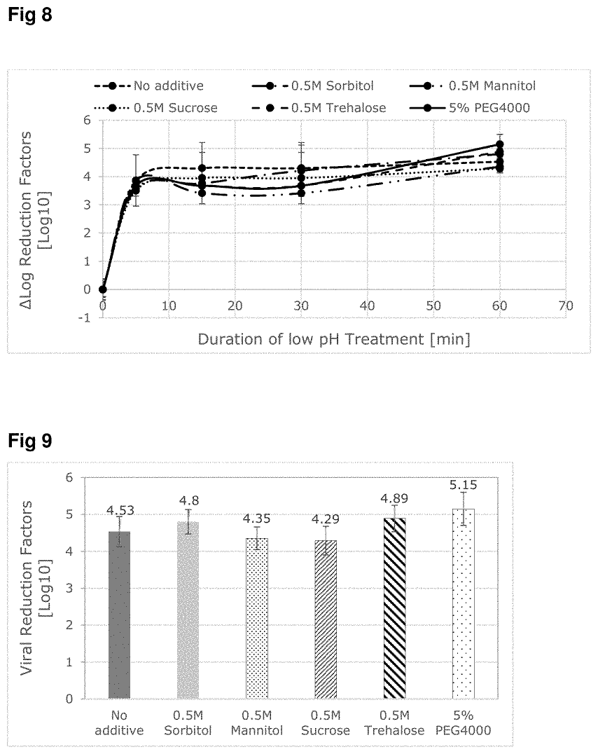

FIG. 8 and FIG. 9 show the results of viral reduction experiments during low pH inactivation hold in the presence of selected neutral excipients (sorbitol, mannitol, sucrose, trehalose and PEG4000) compared to a sample without excipient.

On the basis of the viral reduction factor and an assessment of robustness, a unit operation may be classified as effective, ineffective or moderately effective (FDA QSA, 1998). “Effective” steps provide a reduction factor of at least 4 log 10 and are unaffected by small perturbations in process variables. “Ineffective” steps provide a reduction factor of 1 log 10 or less, and “moderately effective” steps fall between these two extremes (EMD Millipore, 2013).

The usage of the selected excipients still enables an effective virus reduction step (reduction factor of >4 log 10) in all cases with and without selected excipients. This clearly shows that the selected excipients do not negatively affect the viral inactivation process step.

1. A method for purifying a target protein from a cell culture sample, wherein the cell culture sample comprises the target protein, viral compounds and other product and process related impurities, comprising an affinity chromatography step, a virus inactivation step and optionally other purification steps, wherein the affinity chromatography step comprises
a) loading an affinity chromatography column with the cell culture sample thereby binding the target protein to the affinity chromatography column;
b) eluting the target protein from the affinity chromatography column by contacting the affinity chromatography column with an elution buffer having a pH<6 and comprising an excipient, wherein the excipient is selected from the group consisting of disaccharides, polyols and poly (ethylene glycol) polymers;
c) collecting one or more fractions containing the target protein obtained from step (b);
d) combining the fractions obtained from step (c) to form an elution product pool,
and wherein the virus inactivation step comprises
e) incubating the elution product pool at a pH from 2 to 5.

2. The method according to claim 1, wherein the affinity chromatography step is a Protein A affinity chromatography step.

3. The method according to claim 1, wherein the target protein is a monoclonal antibody.

4. The method according to claim 1, wherein the poly (ethylene glycol) polymer has an average molecular weight from 1,000 g/mol to 10,000 g/mol.

5. The method according to claim 1, wherein the excipient is selected from the group consisting of sucrose, trehalose, sorbitol, mannitol and PEG4000.

6. The method according to claim 1, wherein the elution buffer has an excipient concentration from 2% to 15% by weight (in the case of PEG4000) or a concentration in the range of 1 mM to 1.5 M in the solution in the case of disaccharides and polyols.

7. The method according to claim 1, wherein the elution buffer has an excipient concentration from 5% to 10% by weight (in the case of PEG4000) or a concentration in the range of 5 mM to 500 mM in the solution in the case of disaccharides and polyols.

8. The method according to claim 1, wherein the elution buffer is a citrate buffer.

9. The method according to claim 1, wherein the elution buffer has a pH from 2.5 to 5.5.

10. The method according to claim 1, wherein the elution step (b) comprises contacting the affinity chromatography column with the elution buffer using an elution buffer gradient from pH 5.5 to pH 2.75.

11. The method according to claim 1, wherein prior to the incubation step (e) the pH of the elution product pool is adjusted to a pH in the range from pH 2 to pH 5.

12. The method according to claim 1, wherein the incubation step (e) is performed at pH 2.5 to pH 4.5.

13. The method according to claim 1, wherein the incubation step (e) is performed at room temperature.
